(19) United States
(12) Patent Application Publication
(10) Pub. No.: US 2024/0016374 A1
(43) Pub. Date: Jan. 18, 2024

(54) ENDOSCOPE

(71) Applicant: FUJIFILM Corporation, Tokyo (JP)

(72) Inventor: Hiroaki TABE, Kanagawa (JP)

(73) Assignee: FUJIFILM Corporation, Tokyo (JP)

(21) Appl. No.: 18/347,572

(22) Filed: Jul. 6, 2023

(30) Foreign Application Priority Data
Jul. 13, 2022 (JP) 2022-112599

(51) Int. Cl. A61B 1/07; G02B 23/24

(52) U.S. Cl. CPC A61B 1/07; G02B 23/243; G02B 23/2461

(57) ABSTRACT

An illumination optical system that transmits illumination light from a light source via a light guide to a distal end portion of an insertion part to irradiate an observation site from the distal end portion with the illumination light is provided. The illumination optical system includes a first lens located on a distal end side of the light guide and a second lens disposed on a distal end side of the first lens while holding a constant distance interval. An outer diameter of the second lens is larger than an outer diameter of the first lens and is 2.1 mm or more and 3.0 mm or less.

### CROSS-REFERENCE TO RELATED APPLICATIONS

This application claims priority under 35 U. S. C § 119(a) to Japanese Patent Application No. 2022-112599 filed on 13 Jul. 2022. The above application is hereby expressly incorporated by reference, in its entirety, into the present application.

### BACKGROUND OF THE INVENTION

### 1. Field of the Invention

The present invention relates to an endoscope that emits illumination light from a distal end portion of an insertion part.

### 2. Description of the Related Art

Endoscopes are widely used in medical and industrial fields. The endoscope includes an insertion part to be inserted into a subject to be examined and irradiates an observation target with illumination light from a distal end portion of an insertion part. The insertion part of the endoscope is provided with a light guide portion that guides illumination light supplied from a light source device to the distal end portion of the insertion part. The light guide portion guides the illumination light and the observation target is illuminated, whereby the inside of the subject to be examined can be observed.

Generally, the light guide portion includes a light guide composed of an optical fiber bundle, and an illumination optical system that irradiates an observation site with illumination light. Endoscopes described in JP5345171B (corresponding to US2012/0253129A1) and WO2017/130524A comprise an illumination optical system having a three-element configuration in which an optical element having a light reflection function and two convex lenses are combined.

### SUMMARY OF THE INVENTION

Meanwhile, in the endoscopes described in JP5345171B and WO2017/130524A, an intensity distribution at a light emission end is concentrated in the vicinity of a lens optical axis, and a peak value of light density is increased, which may cause coagulation of a substance in a living body, such as blood adhering to a lens surface. In order to avoid this phenomenon, it is necessary to reduce the amount of illumination light guided from a light source device. A decrease in the amount of illumination light may lead to a performance shortfall, such as an inability to achieve sufficient brightness for observation using the endoscope.

An object of the present invention is to provide an endoscope capable of preventing a decrease in an amount of illumination light and reducing a peak value of a light intensity distribution to improve the coagulation resistance of a substance in a living body.

An endoscope of an aspect of the present invention is an endoscope comprising: an illumination optical system that transmits illumination light from a light source via a light guide to a distal end portion of an insertion part to irradiate an observation site from the distal end portion with the illumination light, in which the illumination optical system includes a first lens and a second lens, and an outer diameter of the second lens is larger than an outer diameter of the first lens and is 2.1 mm or more and 3.0 mm or less. The first lens is located on a distal end side of the light guide. The second lens is disposed on a distal end side of the first lens while holding a constant distance interval.

It is preferable that the outer diameter of the second lens is equal to or more than 1.4 times the outer diameter of the first lens. It is preferable that the first lens is a convex lens. It is preferable that the first lens is a biconvex lens.

It is preferable that the second lens is a convex lens. It is preferable that the second lens is a convex-plane lens having a convex shape on a light incidence end side and a planar shape on a light emission end side.

It is preferable that, in a case in which the outer diameter of the second lens is denoted by D and a curvature radius of the second lens on the light incidence end side is denoted by R, the second lens satisfies Conditional Expression: 1.48≤D/R.

It is preferable that an optical element having a light reflection function is disposed between the light guide and the first lens. It is preferable that the optical element includes a light incidence end facing the light guide, an outer peripheral portion having a light reflection function, and a light emission end having a convex shape. It is preferable that the optical element has a total reflection function. It is preferable that the optical element is composed of a core and a clad.

An endoscope of another aspect of the present invention is an endoscope comprising: an illumination optical system that transmits illumination light from a light source via a light guide to a distal end portion of an insertion part to irradiate an observation site from the distal end portion with the illumination light, in which the illumination optical system includes an optical element and a first lens, and a lens outer diameter of the first lens is larger than an outer diameter of the optical element and is 2.1 mm or more and 3.0 mm or less. The optical element includes a light incidence end facing the light guide and an outer peripheral portion having a light reflection function. The first lens is disposed on a distal end side of the optical element while holding a constant distance interval.

It is preferable that the outer diameter of the first lens is equal to or more than 1.4 times the outer diameter of the optical element. It is preferable that the first lens is a concave lens. It is preferable that the first lens is a concave-plane lens having a concave shape on a light incidence end side and a planar shape on a light emission end side.

It is preferable that, in a case in which the outer diameter of the first lens is denoted by D and a curvature radius of the first lens on the light incidence end side is denoted by R, the first lens satisfies Conditional Expression: 2.80≤D/R.

It is preferable that the optical element has a total reflection function. It is preferable that the optical element is composed of a core and a clad.

According to the present invention, it is possible to prevent a decrease in the amount of illumination light and to reduce the peak value of the light intensity distribution to improve the coagulation resistance.

### BRIEF DESCRIPTION OF THE DRAWINGS

FIG. 1 is an external view of an endoscope system.

FIG. 2 is an external perspective view of the endoscope system.

FIG. 3 is a cross-sectional view of a main part of a distal end portion of an insertion part.

FIG. 4 is an exploded perspective view of the distal end portion.

FIG. 5 is an explanatory diagram illustrating a dimensional relationship of an illumination optical system.

FIG. 6 is a graph showing light intensity distributions in a conventional illumination optical system and an illumination optical system of an embodiment of the present invention.

FIG. 7 is a graph showing a light intensity distribution in a case in which ratios of an outer diameter and a curvature radius of a second lens are changed.

FIG. 8 is a graph showing a part that is extracted from the light intensity distribution of FIG. 7.

FIG. 9 is a cross-sectional view of a main part of a distal end portion of an insertion part in a second embodiment.

FIG. 10 is an exploded perspective view of the distal end portion in the second embodiment.

FIG. 11 is an explanatory diagram illustrating a dimensional relationship of an illumination optical system in the second embodiment.

FIG. 12 is a graph showing a light intensity distribution in a case in which ratios of an outer diameter and a curvature radius of a first lens in the second embodiment are changed.

### DESCRIPTION OF THE PREFERRED EMBODIMENTS

### First Embodiment

Outline Configuration of Endoscope System

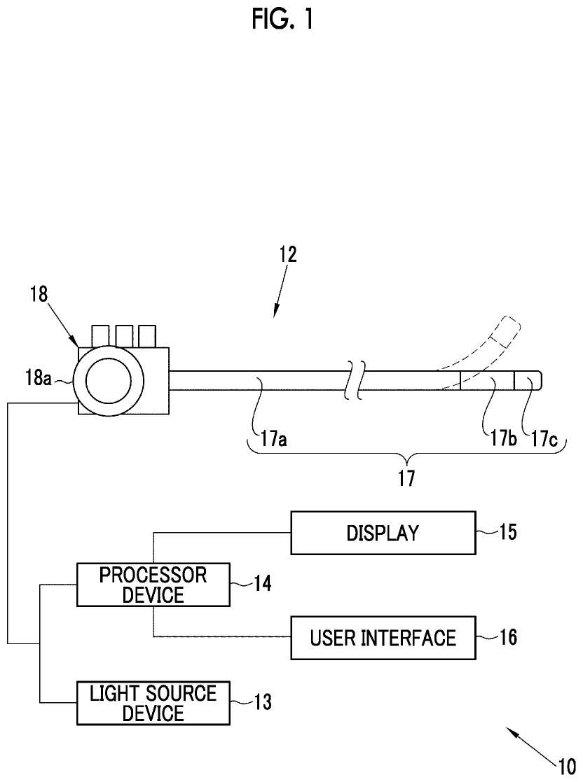

As shown in FIG. 1, an endoscope system 10 includes an endoscope 12, a light source device 13, a processor device 14, a display 15, and a user interface 16. The endoscope 12 is optically and electrically connected to the light source device 13 and is electrically connected to the processor device 14.

Outline Configuration of Endoscope

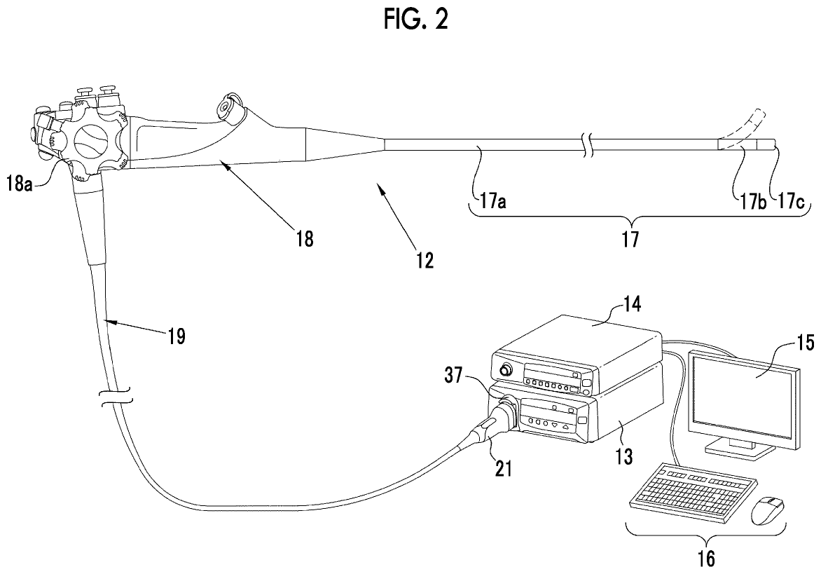

As shown in FIG. 2, the endoscope 12 includes an insertion part 17 to be inserted into a body of a person to be examined, an operation part 18 provided at a proximal end part of the insertion part 17, a universal cable 19 provided at the operation part 18, and an endoscope side connector 21 provided at an end part of the universal cable 19. The endoscope 12 is attachably and detachably connected to a light source device side connector 37 of the light source device 13 via the endoscope side connector 21.

The insertion part 17 is composed of a soft portion 17a, a bendable portion 17b, and a distal end portion 17c, which are consecutively provided in this order from the proximal end to the distal end. By operating an angle knob 18a of the operation part 18, the bendable portion 17b is bent. As a result, the distal end portion 17c faces a desired direction.

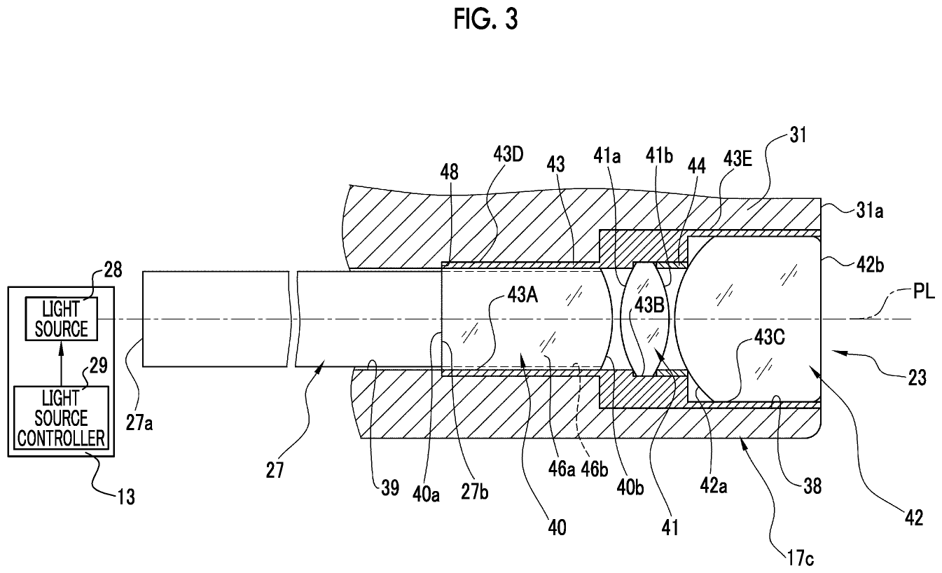

The universal cable 19 is a cable in which a light guide 27 (see FIG. 3) for guiding illumination light emitted by a light source 28, which will be described below, a control line for controlling an imaging element (not shown) provided at the distal end portion 17c of the insertion part 17, a signal line for transmitting an image signal output by the imaging element in a case in which an image of an observation target irradiated with illumination light is captured, a power line for supplying power to each part, such as an imaging sensor, and the like are integrated.

Outline Configuration of Light Source Device and Processor Device

The processor device 14 is electrically connected to the display 15 and the user interface 16. The display 15 outputs and displays an endoscope image of the observation target, which is processed by the processor device 14, information, or the like.

The user interface 16 includes a keyboard, a mouse, a touch pad, a microphone, and the like and has a function of receiving input operations such as function settings. The display 15 outputs and displays an endoscope image or the like of a video or a still image.

Further, the light source device 13 is electrically connected to the processor device 14, and the endoscope side connector 21 of the endoscope 12 is connected to the processor device 14 via the light source device 13. Transmission and reception of the image signal or the like between the light source device 13 and the endoscope side connector 21 is wireless communication. Therefore, the light source device 13 outputs the image signal or the like, which is wirelessly transmitted and received to and from the endoscope side connector 21, to a signal transmission unit (not shown), and the signal transmission unit transmits the image signal to the processor device 14. Further, the light source device 13 supplies power for driving the imaging sensor and the like to the endoscope side connector 21, and this power is also wirelessly supplied.

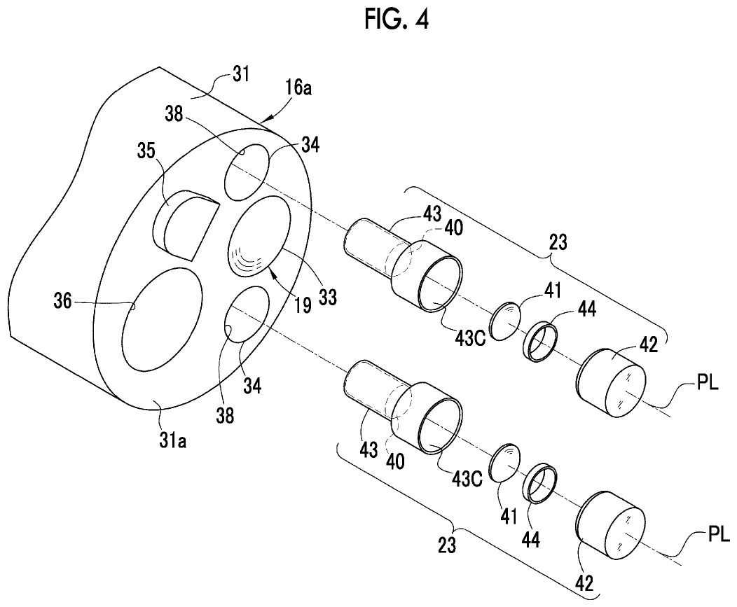

As shown in FIGS. 3 and 4, the light source device 13 comprises the light source 28 and a light source controller 29. The light source 28 emits illumination light used to illuminate the observation target. An illumination optical system 23, which will be described below, is provided at the distal end portion 17c of the endoscope 12. As described above, by connecting the endoscope side connector 21 and the light source device side connector 37, a light incidence end 27a of the light guide 27 of the endoscope 12 faces the light source 28 of the light source device 13. Therefore, the illumination light from the light source 28 is transmitted to the distal end portion 17c of the insertion part 17 via the light guide 27. As a result, the illumination optical system 23 irradiates the observation site with the illumination light from the distal end portion 17c.

The light source controller 29 controls the light source 28. The light source 28 is, for example, a semiconductor light source composed of multiple colors of light emitting diodes (LEDs). The light source controller 29 controls the amount of light emitted from the illumination light by turning on/off the LED and adjusting the driving current and the driving voltage of the LED. The semiconductor light source that constitutes the light source 28 is not limited to the LED and may be a laser diode (LD) or the like. In the following, a case of a normal mode in which white light is emitted as illumination light by the light source 28 will be mainly described.

The distal end portion 17c includes a cylindrical distal end portion main body 31, the above-described imaging element incorporated therein, an objective optical system (not shown), a pair of illumination optical systems 23, and the like. The distal end portion main body 31 is formed of a hard resin, a metal material, or the like. An observation window 33, a pair of illumination windows 34, an air and water supply nozzle 35, and a forceps outlet 36 serving as an outlet of a forceps channel are provided on a distal end surface 31a of the distal end portion main body 31.

The observation window 33 is used to capture an image of a site to be observed, the objective optical system is incorporated in the back thereof, and a part thereof is exposed. The imaging element is disposed in the back of the objective optical system, and the imaging element captures the image of the site to be observed through the objective optical system.

The illumination window 34 is used to illuminate the site to be observed, and the illumination optical system 23 is incorporated in the back thereof. The illumination optical system 23 distributes the illumination light emitted from the light guide 27 so as to be suitable for imaging, for example, so as to uniformly illuminate an imaging range. Although the pair of illumination windows 34 are provided on both sides with the observation window 33 interposed therebetween, the number and the disposition of the illumination windows 34 can be appropriately changed.

The illumination optical system 23 is inserted and assembled into a lens mounting hole 38 formed in the distal end portion main body 31. The lens mounting hole 38 is formed to extend from the distal end surface 31a along an optical axis PL of the illumination optical system 23 toward the proximal end side, and an opening on the distal end surface 31a is the illumination window 34. The lens mounting hole 38 is formed in a shape having a step such that the outer diameter on the distal end side is larger than the outer diameter on the proximal end side in conformity with a lens barrel 43, which will be described below.

In addition, a light guide mounting hole 39 into which the light guide 27 is fitted is formed in the distal end portion main body 31 in the back of the lens mounting hole 38 by being connected to the lens mounting hole 38. Both the lens mounting hole 38 and the light guide mounting hole 39 are each a hole having a circular cross-section and are formed coaxially with each other.

The illumination optical system 23 consists of an optical element 40, a first lens 41, a second lens 42, the lens barrel 43, and a spacer 44. The illumination optical system 23 is inserted into the lens mounting hole 38 in the order of the lens barrel 43 to which the optical element 40 is fixed, the first lens 41, the spacer 44, and the second lens 42.

The lens barrel 43 holds the optical element 40, the first lens 41, and the second lens 42. The lens barrel 43 is fitted into the lens mounting hole 38, whereby the optical element 40, the first lens 41, and the second lens 42 are fixed to the distal end portion main body 31. With this, the optical element 40, the first lens 41, and the second lens 42 are located on the distal end side of the light guide 27.

An inner peripheral surface of the lens barrel 43 includes a first inner diameter part 43A, a second inner diameter part 43B, and a third inner diameter part 43C in this order from the proximal end side to the distal end side. The inner diameter of the first inner diameter part 43A is the same as the outer diameter of the optical element 40, and the inner diameter of the second inner diameter part 43B is larger than the inner diameter of the first inner diameter part 43A and the outer diameter of the optical element 40. With this, light rays emitted from a peripheral edge part of a surface 40a and traveling substantially parallel to the optical axis PL are incident on the first lens 41 without being obstructed.

The inner diameter of the second inner diameter part 43B is slightly larger than the inner diameter of the first inner diameter part 43A and is the same as the outer diameter of the first lens 41. That is, the outer diameter of the first lens 41 is slightly larger than the outer diameter of the optical element 40. With this, the first lens 41 is held by the second inner diameter part 43B without entering the first inner diameter part 43A. That is, the lens barrel 43 functions as a spacer and defines a distance interval between the optical element 40 and the first lens 41. As a result, the first lens 41 is disposed on the distal end side of the optical element 40 while holding a constant distance interval with respect to the optical element 40.

The inner diameter of the third inner diameter part 43C is larger than the inner diameter of the second inner diameter part 43B and is the same as the outer diameter of the second lens 42. That is, the outer diameter of the second lens 42 is larger than the outer diameter of the first lens 41. The spacer 44 has a ring shape and has the same outer diameter as the outer diameter of the first lens 41. The spacer 44 is disposed between the first lens 41 and the second lens 42. With this, the spacer 44 defines a distance interval between the first lens 41 and the second lens 42. That is, the second lens 42 is disposed on the distal end side of the first lens 41 while holding a constant distance interval.

Meanwhile, an outer peripheral surface of the lens barrel 43 includes a first outer diameter part 43D and a second outer diameter part 43E in this order from the proximal end side to the distal end side. As described above, the outer diameter of the first lens 41 is slightly larger than the outer diameter of the optical element 40, and the outer diameter of the second lens 42 is larger than the outer diameter of the first lens 41. Therefore, the outer diameter of the first outer diameter part 43D is one size (corresponding to the thickness of the lens barrel 43) larger than the outer diameter of the optical element 40, and the outer diameter of the second outer diameter part 43E is one size larger than the outer diameter of the second lens 42. That is, the outer peripheral surface of the lens barrel 43 has a shape having a step because the outer diameter of the second outer diameter part 43E is larger than the outer diameter of the first outer diameter part 43D.

The optical element 40 is a rod lens that has a substantially cylindrical shape and that is composed of a core 46a, which is a member of a central part, and a clad 46b, which is formed around the core 46a. The clad 46b has a lower refractive index than the core 46a. The optical element 40 is a convex lens, more specifically, the surface 40a on the light incidence end side, which faces the light guide 27, has a plane, and a surface 40b on the light emission end side (illumination window side) on the opposite side has a convex shape, and this imparts the function of a plano-convex lens.

Further, the optical element 40 guides light to a surface 40b side by reflecting light incident from the surface 40a inside the optical element 40 using a difference in refractive index between the core 46a and the clad 46b, similarly to an optical fiber, and the outer peripheral portion has a light reflection function. The optical element 40 may also be configured by combining a plurality of members so as to have an equivalent function. The optical element 40 may be, for example, an element including a reflecting surface formed on the outer peripheral surface of the plano-convex lens or an element having a total reflection function of reflecting all the light incident on the plano-convex lens.

The lens barrel 43 holds the optical element 40 inside and also functions as a spacer that allows the first lens 41 to be spaced from the optical element 40 at a constant distance interval. The optical element 40 is fixed in the lens barrel 43 such that there is no step between the surface thereof and the end part of the lens barrel 43 on a light guide 27 side, and the optical element is inserted into the lens mounting hole 38 together with the lens barrel 43.

The inner diameter of the light guide mounting hole 39 is slightly smaller (for example, about 0.1 mm) than the lens mounting hole 38. In this way, by coaxially connecting and forming the mounting holes 38 and 39 having different diameters, a contact surface 48 is formed at the boundary between the lens mounting hole 38 and the light guide mounting hole 39 so as to surround the light guide mounting hole 39. An edge of the lens barrel 43 on the light guide side is brought into contact with the contact surface 48 and is locked, whereby the lens barrel 43 and the optical element 40 held therein are positioned. The lens barrel 43 is fixed to the lens mounting hole 38, for example, by curing a thermosetting resin applied between the outer peripheral surface thereof and an inner surface of the lens mounting hole 38.

The outer diameter of the light guide 27 is substantially the same as the inner diameter of the light guide mounting hole 39 and is substantially the same as the outer diameter of the optical element 40. The light guide 27 is fitted and fixed into the light guide mounting hole 39 in a state in which a light emission end 27b is in close contact with the surface 40a of the optical element 40. As a result, all of the illumination light emitted from the light emission end 27b is incident on the surface 40a of the optical element 40 while preventing the outer diameter of the optical element 40 from becoming large.

In the optical element 40, a region of the surface 40a on which effective light is incident is the part of the core 46a, and the diameter of the core 46a is slightly smaller than the outer diameter of the optical element 40. Therefore, the outer diameter of the light guide 27 may be made slightly smaller than the outer diameter of the optical element 40 in conformity with the diameter of the core 46a.

The first lens 41 is a convex lens and is, specifically, a biconvex lens in which a surface 41a on the light incidence end side and a surface 41b on the light emission end side each have a convex shape. The second lens 42 is a convex lens and is, specifically, a convex-plane lens in which a surface 42a on the incidence side of light has a convex shape and a surface 42b on the emission side has a planar shape, and the surface 42b is exposed from the illumination window 34. With such a configuration of the illumination optical system 23, the illumination light is diffused, and the imaging range is uniformly illuminated.

In order to apply the thermosetting resin to the outer peripheral surfaces of the lens barrel 43 and of the second lens 42 and the inner surface of the lens mounting hole 38, for example, the lens barrel 43 and the second lens 42 are each made slightly (for example, about 10 μm) smaller than the inner diameter of the lens mounting hole 38. In addition, a groove in which the thermosetting resin enters may be formed on the outer peripheral surfaces of the lens barrel 43 and of the second lens 42 or on the inner surface of the lens mounting hole 38.

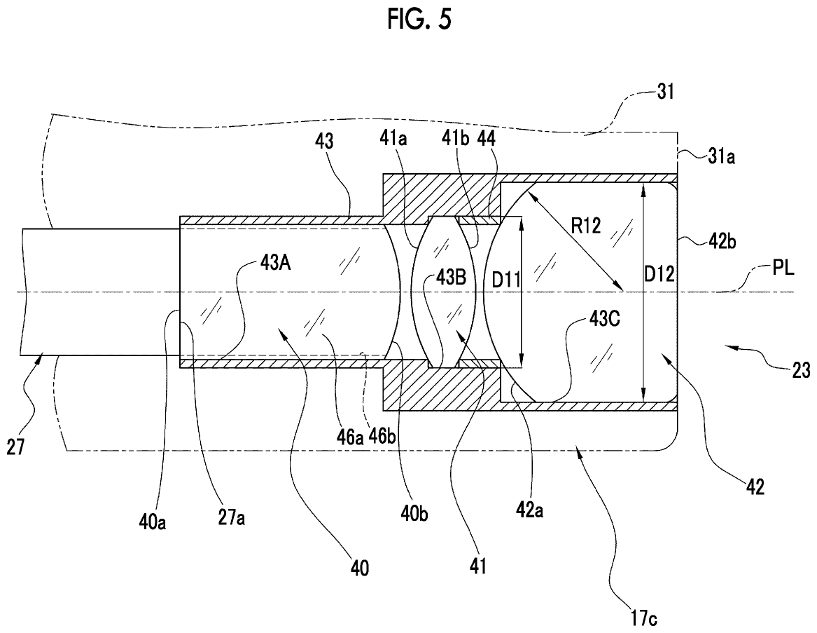

FIG. 5 shows a dimensional relationship between the first lens 41 and the second lens 42. As described above, the outer diameter D12 of the second lens 42 is larger than the outer diameter D11 of the first lens 41. The outer diameter D12 of the second lens 42 is 2.1 mm or more and 3.0 mm or less. In the example shown in FIG. 5, the outer diameter D12 of the second lens 42 is 2.5 mm, the outer diameter D11 of the first lens 41 is 1.7 mm, the curvature radius R12 of the surface 42a (on the incidence side) of the second lens 42 is 1.588 mm, the thickness of the second lens 42 is 2.20 mm, the curvature radius of the surfaces 41a and 41b of the first lens 41 is 1.728 mm, and the curvature radius of the surface 40b (on the emission side) of the optical element 40 is 1.728 mm.

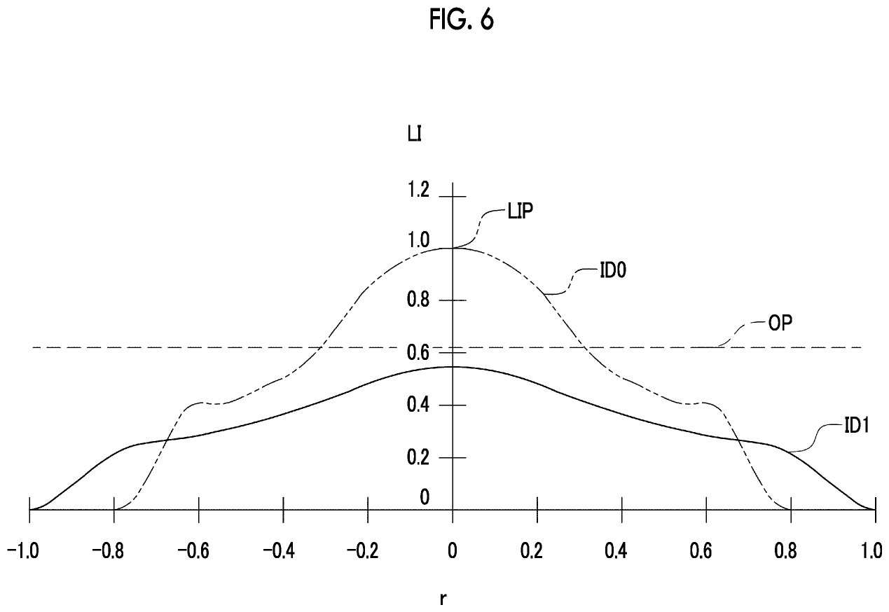

Hereinafter, the reason for defining the outer diameter D12 of the second lens 42 as described above will be described with reference to the graph shown in FIG. 6. A light intensity distribution ID0 indicated by an alternate long and two short dashes line in FIG. 6 shows the light intensity distribution in a case in which it is assumed that the second lens has the same dimensions as the second lens constituting the illumination optical system having a three-element configuration in the conventional endoscope. In this case, it is assumed that the outer diameter of the second lens is 1.7 mm and the diameter of the light emission end excluding the chamfered part is 1.5 mm.

The horizontal axis of the graph shown in FIG. 6 is the radius r from the optical axis PL, and the vertical axis is the light intensity LI of the second lens at the position of the radius r. Further, the light intensity LI uses a relative intensity, which represents the light intensity other than the peak value as a ratio to the peak value, for example, with the peak value LIP of the light intensity distribution ID0 set as 1.0. The light intensity is the density of light within a unit solid angle of a luminous flux.

In a case in which the conventional second lens as shown in FIG. 6 is used, as is clear from the light intensity distribution ID0, the light intensity is concentrated in the vicinity of the lens optical axis, and the peak value LIP greatly protrudes. Therefore, such a lens may cause coagulation of a substance in a living body, such as blood adhering to the lens surface.

In a case in which the distal end portion of the endoscope is immersed in blood and irradiated with the illumination light from the light source 28 for 2 minutes using an illumination optical system composed of a lens having the light intensity distribution ID0, the location where the light intensity exceeding a blood coagulation occurrence point OP shown by the broken line is generated is a location where blood coagulation occurs. As a determination criterion for blood coagulation, it is assumed that blood coagulation has occurred in a case in which the change in the amount of light is 50% or less. From the light intensity distribution ID0, it can be seen that a location where the light intensity distribution ID0 is equal to or more than 0.62 times the peak value is the blood coagulation occurrence point OP.

It can be seen that the peak value of the light intensity distribution needs only be reduced in order to avoid coagulation of a substance in a living body, such as blood adhering to the lens surface. The peak value of the light intensity distribution is inversely proportional to the area of the light emission end. That is, the peak value decreases in a case in which the area of the light emission end is increased. In that respect, in a case in which the outer diameter of the light emission end (excluding the chamfered part) of the second lens 42 is denoted by D120 and the peak value of the light density is denoted by LIP, Expression (1) needs only be satisfied.

LIP=(1.5)²/(D120)²≤0.62 (1)

From Expression (1), D120 is 1.9 mm or more. In consideration of the chamfered part (0.1 mm on one side) of the outer peripheral edge of the lens, the lens outer diameter D12 of the second lens 42 is D120+0.2 mm, that is, the outer diameter D12 is 2.1 mm or more. Further, in consideration of the outer diameter and the internal space of the distal end portion 17c of the endoscope 12, and the like, the outer diameter D12 of the second lens 42 is limited to 3.0 mm. From the above, the outer diameter of the second lens 42 is 2.1 mm or more and 3.0 mm or less.

A light intensity distribution ID1 indicated by a solid line in FIG. 6 shows a light intensity distribution in a case in which it is assumed that the outer diameter of the second lens 42 is 2.1 mm or more and 3.0 mm or less. As shown in the light intensity distribution ID1, the peak value of the light density decreases, and the entire light intensity distribution ID1 is below the blood coagulation occurrence point OP.

Further, the outer diameter D12 of the second lens 42 is equal to or more than 1.4 times the outer diameter D11 of the first lens 41. From the viewpoint of transmission efficiency and processing accuracy, the outer diameter D11 of the first lens 41 needs to be at least equal to or more than the optical element 40, and the outer diameter of the optical element 40 needs to be 1.5 mm or more. In that respect, in order to set the outer diameter D11 of the first lens 41 to 1.5 mm or more and further to set the outer diameter D12 of the second lens 42 to 2.1 mm or more as described above, it is necessary to satisfy a condition that the outer diameter D12 of the second lens 42 is equal to or more than 1.4 times the outer diameter D11 of the first lens 41.

Further, in a case in which the outer diameter of the second lens 42 is denoted by D12 and the curvature radius of the second lens 42 on the light incidence end side is denoted by R12, the second lens 42 satisfies Expression (2).

1.48≤D12/R12 (2)

The reason for satisfying Expression (2) will be described below. In a case in which the outer diameter R2 of the second lens 42 is increased, D12/R12 decreases, and the illumination light distribution narrows (the light intensity at the peripheral part decreases).

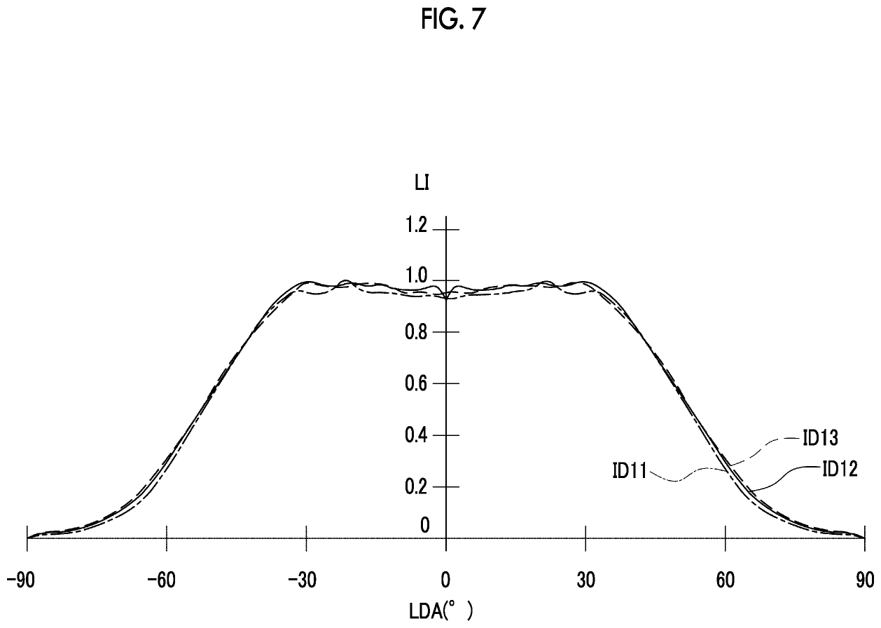

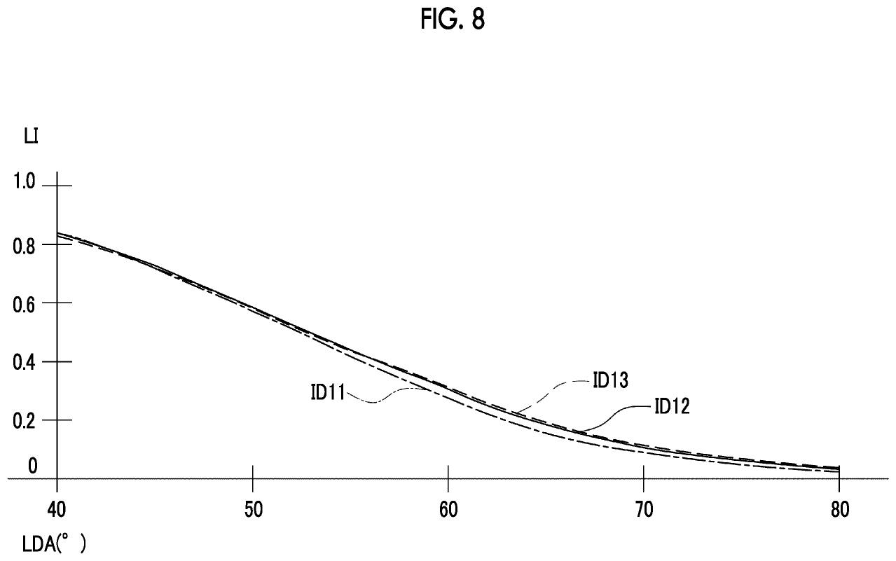

The light intensity distributions ID11 to ID13 shown in FIG. 7 show the light intensity distribution in a case in which the outer diameter R2 of the second lens 42 is changed, and the horizontal axis represents the light distribution angle LDA, and the vertical axis represents the light intensity LI with respect to the light distribution angle. In addition, FIG. 8 shows a part corresponding to a light distribution angle of 40° to 80°, which is extracted from the light intensity distributions ID to ID13. The light intensity is a relative intensity, which represents the light intensity other than the peak value as a ratio to the peak value, for example, with the peak value of each light intensity distribution set as 1.0. The light intensity is the density of light within a unit solid angle of a luminous flux. A light intensity distribution ID shows a case in which D12/R12 is 1.25, the light intensity distribution ID12 shows a case in which D12/R12 is 1.48, and the light intensity distribution ID13 shows a case in which D12/R12 is 1.57.

The illumination light distributions are substantially the same between the light intensity distribution ID12 and the light intensity distribution ID13, that is, there is no difference in light intensity even at the peripheral part (the light distribution angle is 40° or more). On the other hand, in the light intensity distribution ID11 and the light intensity distribution ID12, the illumination light distribution of the light intensity distribution ID11 is narrow, and the light intensity at the peripheral part (the light distribution angle is 40° or more) is small. Therefore, in the case of the light intensity distribution ID11 in which D12/R12 is less than 1.48, the peripheral part of the screen in the captured image of the endoscope 12 is darkened. On the other hand, in the case of the light intensity distributions ID12 and ID13 in which D12/R12 is 1.48 or more, the peripheral part of the screen in the captured image of the endoscope 12 is brightened as described above.

As described above, in the present embodiment, since the outer diameter D12 of the second lens 42 is larger than the outer diameter D11 of the first lens 41 and is set to 2.1 mm or more and 3.0 mm or less, the peak value of the light intensity can be reduced to improve the coagulation resistance of a substance in a living body. In addition, by reducing the peak value of the light intensity, it is possible to transmit illumination light without reducing the amount of illumination light guided from the light source device, so that sufficient brightness can be obtained in observation with the endoscope 12. Further, since the outer diameter D12 of the second lens 42 is equal to or more than 1.4 times the outer diameter D11 of the first lens 41, it is possible to more reliably reduce the peak value of the light intensity.

In addition, since the outer diameter of the second lens 42 needs only be increased and the optical element 40 and, as the optical element 40 and the first lens 41, those of the conventional illumination optical system can be used, it is possible to suppress an increase in cost. By designing basic optical performance such as lens light transmittance and illumination light distribution equivalent to those of the conventional illumination optical system except for the outer diameter of the second lens 42, the second lens 42 can be used without degradation in performance.

In addition, by satisfying 1.48≤D12/R12 for the outer diameter D12 and the curvature radius R12 of the second lens 42, it is possible to prevent a decrease in light intensity at the peripheral part. Therefore, even the peripheral part of the screen in the captured image of the endoscope 12 is sufficiently brightened, and a good observation environment can be obtained.

### Second Embodiment

In the above-described first embodiment, although the illumination optical system 23 having a three-element configuration is provided in which the optical element 40, the first lens 41, and the second lens 42 are provided, the present invention is not limited thereto, and in a second embodiment, which will be described below, an illumination optical system having a two-element configuration is provided in which an optical element and a first lens are provided. The same components and the like as those of the above-described first embodiment are designated by the same reference numerals, and the description thereof will be omitted.

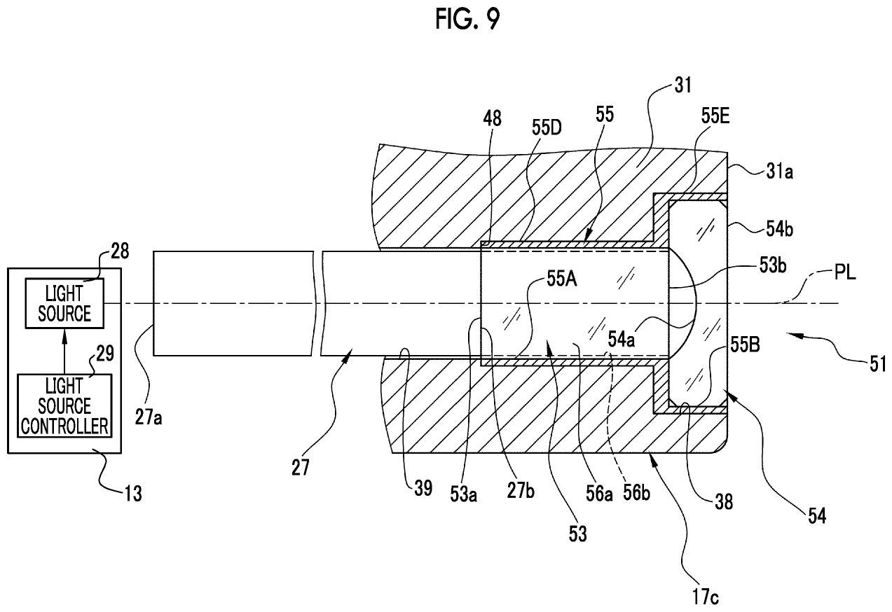

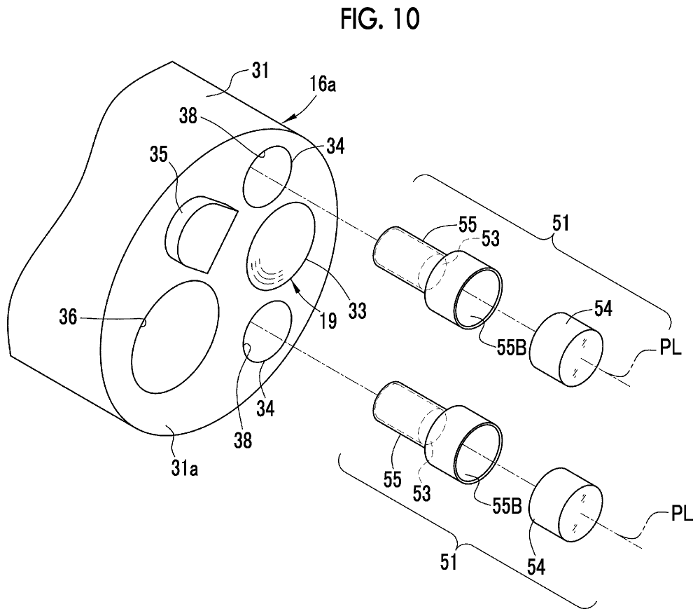

As shown in FIGS. 9 and 10, an illumination optical system 51 is inserted and assembled into the lens mounting hole 38 formed in the distal end portion main body 31. The illumination optical system 51 consists of an optical element 53, a first lens 54, and a lens barrel 55. The illumination optical system 51 is inserted into the lens mounting hole 38 in the order of the lens barrel 55 to which the optical element 53 is fixed and the first lens 54. That is, the illumination optical system 51 is located at the distal end portion 17c of the insertion part 17, similarly to the illumination optical system 23 in the first embodiment. Since the illumination light from the light source 28 is transmitted to the distal end portion 17c via the light guide 27, the illumination optical system 51 irradiates the observation site with the illumination light from the distal end portion 17c.

The lens barrel 55 holds the optical element 53 and the first lens 54. The lens barrel 55 is fitted into the lens mounting hole 38, whereby the optical element 53 and the first lens 54 are fixed to the distal end portion main body 31. With this, the optical element 53 and the first lens 54 are located on the distal end side of the light guide 27.

An inner peripheral surface of the lens barrel 55 includes a first inner diameter part 55A and a second inner diameter part 55B in this order from the proximal end side to the distal end side. The inner diameter of the first inner diameter part 55A is the same as the outer diameter of the optical element 53, and the inner diameter of the second inner diameter part 55B is larger than the inner diameter of the first inner diameter part 55A and the outer diameter of the optical element 53. With this, light rays emitted from a peripheral edge part of a surface 53a and traveling substantially parallel to the optical axis PL are incident on the first lens 54 without being obstructed.

The inner diameter of the second inner diameter part 55B is larger than the inner diameter of the first inner diameter part 55A and is the same as the outer diameter of the first lens 54. That is, the outer diameter of the first lens 54 is larger than the outer diameter of the optical element 53. With this, the first lens 54 is held by the second inner diameter part 55B without entering the first inner diameter part 55A.

Meanwhile, an outer peripheral surface of the lens barrel 55 includes a first outer diameter part 55D and a second outer diameter part 55E in this order from the proximal end side to the distal end side. As described above, the outer diameter of the first lens 41 is larger than the outer diameter of the optical element 40. Therefore, the outer diameter of the first outer diameter part 55D is one size (corresponding to the thickness of the lens barrel 55) larger than the outer diameter of the optical element 53, and the outer diameter of the second outer diameter part 55E is one size larger than the outer diameter of the first lens 54. That is, the outer peripheral surface of the lens barrel 55 has a shape having a step because the outer diameter of the second outer diameter part 55E is larger than the outer diameter of the first outer diameter part 55D.

Similarly to the optical element 40 in the first embodiment, the optical element 53 is a rod lens that has a substantially cylindrical shape and that is composed of a core 56a, which is a member at a central part, and a clad 56b, which is formed around the core 56a. The clad 46b has a lower refractive index than the core 46a. In the optical element 53, both the surface 53a on the light incidence end side, which faces the light guide 27, and a surface 53b on the light emission end side (illumination window side) on the opposite side are planes.

Further, similarly to the optical element 40 in the first embodiment, the optical element 53 guides light to a surface 53b side by reflecting light incident from the surface 53a inside the optical element 53 using a difference in refractive index between the core 56a and the clad 56b, similarly to an optical fiber, and the outer peripheral portion has a light reflection function. The optical element 53 may have a total reflection function of reflecting all incident light.

The first lens 54 is a concave lens and is, specifically, a concave-plane lens in which a surface 54a on the light incidence end side has a concave shape and a surface 54b on the light emission end side has a planar shape, and the surface 54b is exposed from the illumination window 34. With such a configuration of the illumination optical system 51, the illumination light is diffused, and the imaging range is uniformly illuminated.

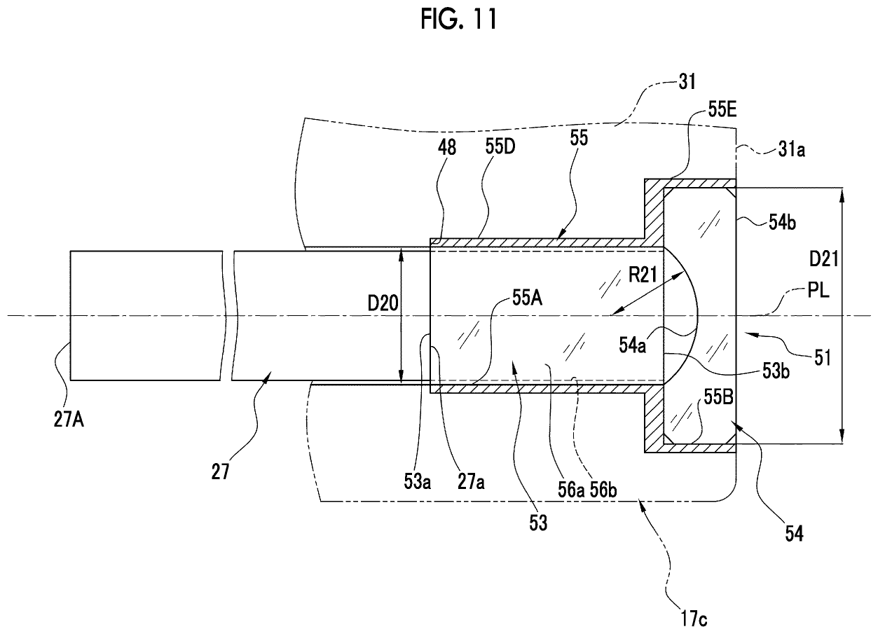

FIG. 11 shows a dimensional relationship between the optical element 53 and the first lens 54. As described above, the outer diameter D21 of the first lens 54 is larger than the outer diameter D20 of the optical element 53. The outer diameter D21 of the first lens 54 is 2.1 mm or more and 3.0 mm or less. In the example shown in FIG. 11, the outer diameter D21 of the first lens 54 is 2.5 mm, the outer diameter D20 of the optical element 53 is 1.5 mm, the curvature radius R21 of the surface 54a (on the incidence side) of the first lens 54 is 0.892 mm, and the thickness of the first lens 54 is 0.75 mm.

The reason for defining the outer diameter D21 of the first lens 54 as described above is the same as the reason for defining the outer diameter D12 of the second lens 42 in the first embodiment. In the first lens constituting the conventional illumination optical system having a two-element configuration, the outer diameter is 1.7 mm, and the diameter of the light emission end excluding the chamfered part is about 1.5 mm.

In such a conventional first lens, the light intensity is concentrated in the vicinity of the lens optical axis, and the peak value greatly protrudes. Therefore, it is can be seen that the peak value of the light intensity distribution needs only be reduced in order to avoid coagulation of the substance in the living body as in the first embodiment. The peak value of the light intensity distribution is inversely proportional to the area of the light emission end. That is, the peak value decreases in a case in which the area of the light emission end is increased. In that respect, in a case in which the outer diameter of the light emission end (excluding the chamfered part) of the first lens 54 is denoted by D210 and the peak value of the light density is denoted by LIP, Expression (3) needs only be satisfied.

LIP=(1.5)²/(D210)²≤0.62 (3)

From Expression (3), D210 is 1.9 mm or more. In consideration of the chamfered part (0.1 mm on one side) of the outer peripheral edge of the lens, the lens outer diameter D21 of the first lens 54 is D210+0.2 mm, that is, the outer diameter D21 is 2.1 mm or more. Further, in consideration of the outer diameter and the internal space of the distal end portion 17c of the endoscope 12, and the like, the outer diameter D21 of the first lens 54 is limited to 3.0 mm. From the above, the outer diameter of the first lens 54 is set to 2.1 mm or more and 3.0 mm or less.

Further, the outer diameter D21 of the first lens 54 is equal to or more than 1.4 times the outer diameter D11 of the optical element 53. From the viewpoint of transmission efficiency and processing accuracy, the optical element 53 needs to have an outer diameter of 1.5 mm or more. In that respect, in order to set the outer diameter D20 of the optical element 53 to 1.5 mm or more and further to set the outer diameter D21 of the first lens 54 to 2.1 mm or more as described above, it is necessary to satisfy a condition that the outer diameter D21 of the first lens 54 is equal to or more than 1.4 times the outer diameter D11 of the first lens 41.

Further, in a case in which the outer diameter of the first lens 54 is denoted by D21 and the curvature radius of the first lens 54 on the light incidence end side is denoted by R21, the first lens 54 satisfies Expression (4).

2.80≤D21/R21 (4)

The reason for satisfying Expression (4) will be described below. In a case in which the outer diameter R21 of the first lens 54 is increased, D21/R21 decreases, and the illumination light distribution narrows (the light intensity at the peripheral part decreases).

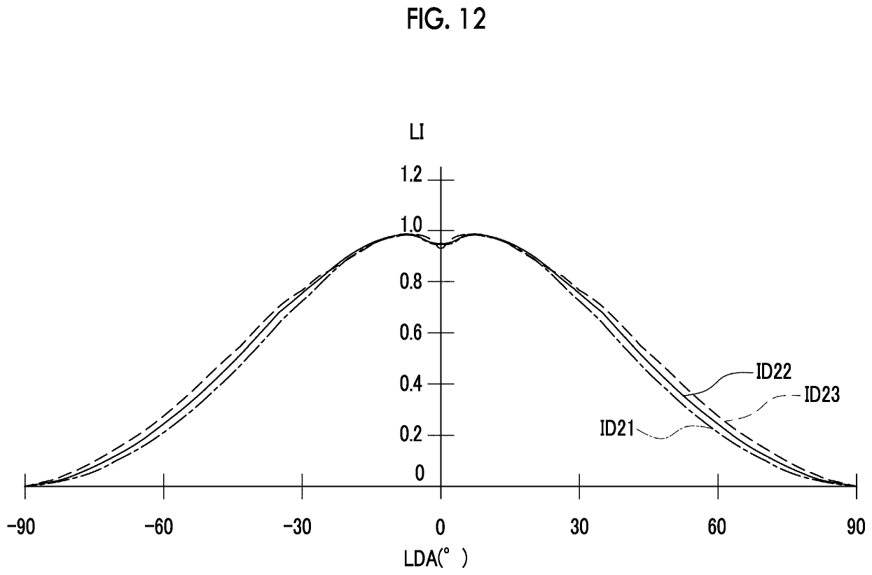

The light intensity distributions ID21 to ID23 shown in FIG. 12 show the light intensity distribution in a case in which the outer diameter R21 of the first lens 54 is changed, and the horizontal axis represents the light distribution angle LDA, and the vertical axis represents the light intensity LI with respect to the light distribution angle. The light intensity is a relative intensity, which represents the light intensity other than the peak value as a ratio to the peak value, for example, with the peak value of each light intensity distribution set as 1.0. The light intensity is the density of light within a unit solid angle of a luminous flux. The light intensity distribution ID21 shows a case in which D21/R21 is 2.50, the light intensity distribution ID22 shows a case in which D21/R21 is 2.63, and the light intensity distribution ID23 shows a case in which D21/R21 is 2.80.

In the light intensity distribution ID22 and the light intensity distribution ID23, the illumination light distribution of the light intensity distribution ID22 is narrow, and the light intensity at the peripheral part (the light distribution angle is 40° or more) is small. Further, in the light intensity distribution ID21 and the light intensity distribution ID22, the illumination light distribution of the light intensity distribution ID21 is even narrower, and the light intensity at the peripheral part (the light distribution angle is 40° or more) is even smaller. Therefore, in the light intensity distributions ID21 and ID22 in which D21/R21 is less than 2.80, the peripheral part of the screen in the captured image of the endoscope 12 is darkened. On the other hand, in the case of the light intensity distribution ID23 in which D21/R21 is 2.80 or more, the peripheral part of the screen in the captured image of the endoscope 12 is brightened as described above.

As described above, in the present embodiment, since the outer diameter D21 of the first lens 54 is larger than the outer diameter D20 of the optical element 53 and is 2.1 mm or more and 3.0 mm or less, the peak value of the light intensity can be reduced to improve the coagulation resistance of the substance in the living body. In addition, by reducing the peak value of the light intensity, it is possible to transmit illumination light without reducing the amount of illumination light guided from the light source device, so that sufficient brightness can be obtained in observation with the endoscope. Further, since the outer diameter D21 of the first lens 54 is equal to or more than 1.4 times the outer diameter D20 of the optical element 53, it is possible to more reliably reduce the peak value of the light intensity.

In addition, by satisfying 2.80≤D21/R21 for the outer diameter D21 and the curvature radius R21 of the first lens 54, it is possible to prevent a decrease in light intensity at the peripheral part. Therefore, even the peripheral part of the screen in the captured image of the endoscope is sufficiently brightened, and a good observation environment can be obtained.

In each of the above-described embodiments, the illumination optical system in which the diameter of the second lens 42 and the first lens 54 is increased, that is, the illumination optical systems 23 and 51, is applied to both of the pair of illumination optical systems, but the present invention is not limited thereto, and the illumination optical systems 23 and 51 may be used as only one of the pair of illumination optical systems, and an illumination optical system using the conventional second lens and first lens may be used as the other. In this case, in a case in which a configuration is employed in which a liquid is jetted from the air and water supply nozzle 35 to the illumination optical system using the conventional second lens and first lens, it is possible to reduce the likelihood of the coagulation of the substance in the living body.

In the above-described embodiment, a medical endoscope has been described as an example, but the present invention can also be applied to, for example, an endoscope or the like used for other applications such as industrial applications. In addition, in the above-described embodiment, although two illumination optical systems and two light guides are provided as the light guide portion, the present invention is not limited thereto, and three or more illumination optical systems and three or more light guides may be provided.

### EXPLANATION OF REFERENCES

10: endoscope system
12: endoscope
13: light source device
14: processor device
15: display
16: user interface
17: insertion part
17a: soft portion
17b: bendable portion
17c: distal end portion
18: operation part
18a: angle knob
19: universal cable
21: endoscope side connector
22: objective optical system
23, 51: illumination optical system
27: light guide
27a: light incidence end
27b: light emission end
28: light source
29: light source controller
31: distal end portion main body
31a: distal end surface
33: observation window
34: illumination window
air and water supply nozzle
36: forceps outlet
37: light source device side connector
38: lens mounting hole
39: light guide mounting hole
40: optical element
40a: surface
40b: surface
41: first lens
41a, 41b: surface
42: second lens
42a, 42b: surface
43: lens barrel
43A: first inner diameter part
43B: second inner diameter part
43C: third inner diameter part
43D: first outer diameter part
43E: second outer diameter part
44: spacer
46a: core
46b: clad
48: contact surface
53: optical element
53a, 53b: surface
54: first lens
54a, 54b: surface
55: lens barrel
55A: first inner diameter part
55B: second inner diameter part
55D: first outer diameter part
55E: second outer diameter part
56a: core
56b: clad
D11, D12, D20, D21: outer diameter
R12, R21: curvature radius
r: radius
ID0, ID1, ID11, ID12, ID13, ID21, ID22, ID23: light intensity distribution
LDA: light distribution angle
LI: light intensity
LIP: peak value
OP: blood coagulation occurrence point

What is claimed is:

1. An endoscope comprising:
an illumination optical system that transmits illumination light from a light source via a light guide to a distal end portion of an insertion part to irradiate an observation site from the distal end portion with the illumination light,
wherein the illumination optical system includes:
a first lens located on a distal end side of the light guide; and
a second lens disposed on a distal end side of the first lens while holding a constant distance interval, and
wherein an outer diameter of the second lens is larger than an outer diameter of the first lens and is 2.1 mm or more and 3.0 mm or less.

2. The endoscope according to claim 1,
wherein the outer diameter of the second lens is equal to or more than 1.4 times the outer diameter of the first lens.

3. The endoscope according to claim 2,
wherein the first lens is a convex lens.

4. The endoscope according to claim 3,
wherein the first lens is a biconvex lens.

5. The endoscope according to claim 4,
wherein the second lens is a convex lens.

6. The endoscope according to claim 5,
wherein the second lens is a convex-plane lens having a convex shape on a light incidence end side and a planar shape on a light emission end side.

7. The endoscope according to claim 6,
wherein, in a case in which the outer diameter of the second lens is denoted by D and a curvature radius of the second lens on the light incidence end side is denoted by R, the second lens satisfies a following conditional expression:
1.48≤D/R.

8. The endoscope according to claim 7,
wherein an optical element having a light reflection function is disposed between the light guide and the first lens.

9. The endoscope according to claim 8,
wherein the optical element includes a light incidence end facing the light guide, an outer peripheral portion having a light reflection function, and a light emission end having a convex shape.

10. The endoscope according to claim 9,
wherein the optical element has a total reflection function.

11. The endoscope according to claim 10,
wherein the optical element is composed of a core and a clad.

12. An endoscope comprising:
an illumination optical system that transmits illumination light from a light source via a light guide to a distal end portion of an insertion part to irradiate an observation site from the distal end portion with the illumination light,
wherein the illumination optical system includes:
an optical element that includes a light incidence end facing the light guide and an outer peripheral portion having a light reflection function; and
a first lens disposed on a distal end side of the optical element while holding a constant distance interval, and
wherein a lens outer diameter of the first lens is larger than an outer diameter of the optical element and is 2.1 mm or more and 3.0 mm or less.

13. The endoscope according to claim 12,
wherein the outer diameter of the first lens is equal to or more than 1.4 times the outer diameter of the optical element.

14. The endoscope according to claim 13,
wherein the first lens is a concave lens.

15. The endoscope according to claim 14,
wherein the first lens is a concave-plane lens having a concave shape on a light incidence end side and a planar shape on a light emission end side.

16. The endoscope according to claim 15,
wherein, in a case in which the outer diameter of the first lens is denoted by D and a curvature radius of the first lens on the light incidence end side is denoted by R, the first lens satisfies a following conditional expression:
2.80≤D/R.

17. The endoscope according to claim 16,
wherein the optical element has a total reflection function.

18. The endoscope according to claim 17,
wherein the optical element is composed of a core and a clad.
